(19) United States
(12) Patent Application Publication
(10) Pub. No.: US 2007/0088385 A1
(43) Pub. Date: Apr. 19, 2007

(54) Vibrating tourniquet

(76) Inventor: Eric S. Perry, Troy, MI (US)

Correspondence Address: Steven W. Hays, Suite 250, 28333 Telegraph Road, Southfield, MI, 48034, US

(21) Appl. No.: 11/251,047

(22) Filed: Oct. 14, 2005

(51) Int. Cl. A61B 17/00

(52) U.S. Cl. 606/203

(57) ABSTRACT

A vibrating tourniquet is disclosed in which one or more vibrating devices are coupled within a pliable strap portion and are activated to interrupt nerve transmissions, and supply an alternative stimulus, that decreases the sensation of pain as the needle or IV are introduced through the skin and into a vein. The vibration devices are electrically coupled to a controller mechanism and the controller mechanism may be turned on or off after the tourniquet is applied to the arm. In alternative versions, the frequency and amplitude of the vibration may be selectively increased or decreased cooperatively or separately by adjusting the controller mechanism as needed.

### TECHNICAL FIELD

The present invention relates to a tourniquet, more particularly to a vibrating tourniquet.

### BACKGROUND OF THE INVENTION

Tourniquets are used in medical emergencies to arrest life-threatening arterial or venous bleeding. A tourniquet is applied around an arm or leg, or other body extremity, to constrict the blood vessels between the patient's heart and the bleeding wound. Tourniquets must be used with care and knowledge, as stopping the flow of blood to the wound also stops the flow of blood to the entire extremity below the tourniquet; prolonged blood deprivation from improperly extended or regulated tourniquet use can lead to tissue death in the affected limb.

Besides the first-aid application for stopping the bleeding, a tourniquet can also be used be used for intravenous injection (“IV” or angiocatheter) or blood drawing, in which the tourniquet is applied to a limb. The purpose of the tourniquet is to engorge the veins distal to the tourniquet, thereby making it easier to place the catheter or needle.

When a tourniquet is used to assist injection, the traditional tourniquet consisting of a slim circular rubber tube is used. In general, the circular rubber tube is stretched to a fixed position for its application. The stretching process causes a tugging action on the skin that may result in discomfort to the patient. In addition, the patients feel a sensation of pain and anxiety as the needle or IV is being introduced through the skin. Studies have shown that most patients describe the IV as being very painful, and remember it as the most traumatic and anxious aspect of their procedure.

There is a need for decreasing the pain and anxiety associated with blood draws and IV placements.

### SUMMARY OF THE INVENTION

The improved tourniquet of the present invention introduces one or more vibrating devices within its pliable strap portion that interrupt nerve transmissions, and supply an alternative stimulus that decreases the sensation of pain as the needle or IV are introduced through the skin and into a vein. The devices contain a controller mechanism that may be turned on or off after the tourniquet is applied to the arm.

In alternative version, the frequency and amplitude of the vibration may be selectively increased or decreased by adjusting the controller mechanism as needed.

In another alternative version, the frequency and amplitude of the vibration may be separately selectively increased or decreased by adjusting the controller mechanism as needed.

Other objects and features of the present invention will become apparent when viewed in light of the detailed description of the preferred embodiment when taken in conjunction with the attached drawings and appended claims.

### BRIEF DESCRIPTION OF THE DRAWINGS

FIG. 1 is a perspective view of a vibrating tourniquet according to one preferred embodiment of the present invention;

FIG. 2 is a perspective view of the vibrating tourniquet of FIG. 1 coupled to the limb of a patient;

FIG. 3 is a perspective view of a vibrating tourniquet according to one preferred embodiment of the present invention; and

FIG. 4 is a perspective of the vibrating tourniquet of FIG. 3 coupled to the limb of a patient.

### DETAILED DESCRIPTION OF THE PREFERRED EMBODIMENTS

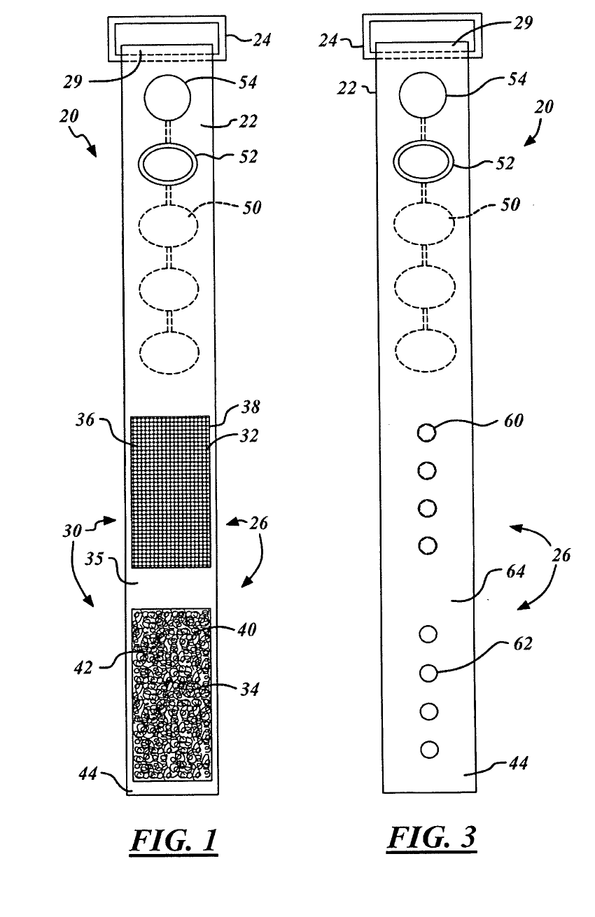

Referring now to FIGS. 1 and 3, two preferred versions of a vibrating tourniquet 20 is illustrated having a strap portion 22. The strap portion 22 includes a fastening loop 24 at one end and a fastening mechanism 26 closer to its other end.

The strap portion 22 is formed of a pliable, or otherwise flexible, material. Preferred materials used in the strap portion include natural materials such rubber or manufactured fabrics. The fastening loop 24 is formed of hard rubber, plastic or metal and is retained within a looped end portion 28 of the strap portion 22. Alternatively, the fastening loop 24 may be secured to the end portion 29 of the strap by an adhesive or other means well known to those of ordinary skill in the art.

Coupled to the strap portion 22 between the fastening mechanism 26 and the fastening loop 24 are one or more vibrations devices 50 electrically coupled to a controller mechanism 52. The vibration devices 50 and controller mechanism 52. The controller mechanism 50 and vibration devices are also electrically coupled to an electrical power source such as a battery 54 or wall outlet (not shown). Preferably, the vibration devices 50, controller mechanism 52, and battery 54 are contained within the interior of the strap 22, although they may also be coupled to an outer surface of the strap 22.

The vibration devices 50 vibrate at a preset amplitude and frequency as defined by the controller mechanism 52. The controller mechanism 52 may be a simple on/off type controller, which provides an electrical signal to the vibration device 50 to vibrate at a single preset frequency and amplitude. The controller mechanism 52 is thus controlled by an operator by simply placing the controller mechanism in an on position, preferably using a push or squeeze method, a switch, a dial, or some other method well known to a person of ordinary skill in the art.

Alternatively, the controller mechanism 52 may have a plurality of different settings, (i.e. a multi-position controller mechanism), thereby allowing the vibration mechanism 52 to vibrate at a variety of different amplitudes and frequencies. The controller mechanism 52 may therefore be in the form of a dial, a multi-position switch, a multi-position pull or squeeze actuation device, or any other multi-position control mechanism 52 known to those of ordinary skill in the art. Further, the controller mechanism may have separate controllers, in the form of dials or the like for the frequency and amplitude of vibration, hereinafter called a multiple control

The fastening mechanism 26, as shown in the preferred embodiment of FIG. 1, is in the form of a hook and loop fastening mechanism 30 having a hook portion 32 and a loop portion 34 separated by a middle fabric portion 35. The hook portion 32 has a plurality of hooks 36 coupled to and extending away from a fabric backing material 38. The loop portion 34 includes a plurality of loops 40 coupled to and extending away from a fabric backing material 42. The fabric backing materials 38, 42 are preferably sewn, along its outer periphery, to the underlying strap 22 such that the respective hooks 36 or loops 40 extend upward and away from the backing material 38, 42 and strap portion 22. Alternatively, the fabric backing materials 38, 42 may be glued or otherwise adhered to the strap portion 32 by methods well known to those of ordinary skill in the art. While the embodiment as shown in FIG. 1 illustrates the loop portion 34 being closer to the free end 44 of the tourniquet 20 than the hook portion 32, the positioning of the hook portion 32 and loop portion 34 relative to the free end 44 are inconsequential to the use of the tourniquet and thus are interchangeable.

The fastening mechanism 26, as shown in the preferred embodiment of FIG. 3, consists of a series of pegs 60 coupled to the fabric portion 22 and an equal number of correspondingly sized and shaped holes 62 extending through the fabric portion 22. A middle portion 64 of the fabric 22 separates the pegs 60 and holes 62. The holes 62 are preferably located at the free end 44 of the fabric portion 22.

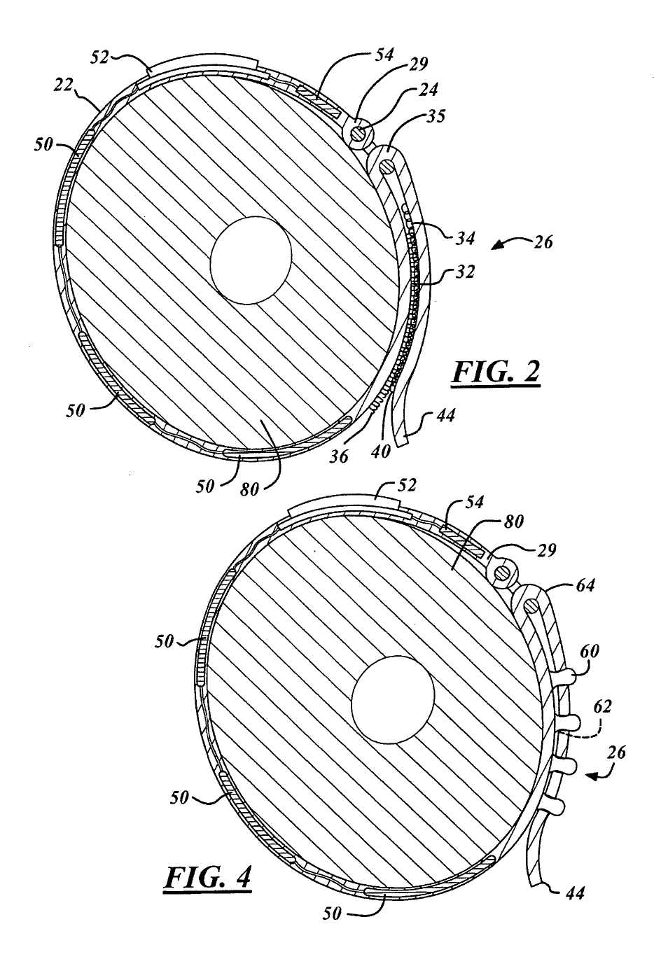

To utilize the vibrating tourniquet 20 of FIG. 1, as shown best in FIGS. 2, the tourniquet 20 is first fastened around the appendage, here the upper arm 80, of a patient and secured. This is accomplished by first introducing the free end 44 of the fabric portion 22 through the fastening loop 24. Next, the free end 44 is bent over the fastening loop 24 such that the hook portion 32 is closely coupled to the loop portion 34 and such that the strap 22 is tight enough to dilate the veins in the patient's arm 80 and such that the vibrations devices 50 are substantially flush against the user's arm 80. The hook portion 32 is then pressed onto the loop portion, or vice-versa such that the plurality of hooks 36 reversibly interlock with the respective loops 40.

For the vibrating tourniquet of FIG. 2, as shown best in FIG. 4, the tourniquet 20 is first fastened around the appendage, here the upper arm 80, of a patient and secured. This is accomplished by first introducing the free end 44 of the fabric portion 22 through the fastening loop 24. Next, the free end 44 is bent over the fastening loop 24 such that the holes 62 are closely coupled to the loop portion 34 and such that the strap 22 is tight enough to dilate the veins in the patient's arm 80 and such that the vibrations devices 50 are substantially flush against the user's arm 80. At least one of the pegs 60 is then pressed through a respective hole 62.

Next, the controller mechanism 52 is actuated to send an electrical signal to the respective one or more vibration devices 50, which then vibrate at a desired frequency and amplitude as a function of the sent electrical signal. As discussed above, the type of actuation is dependent upon the type of controller mechanism 52 utilized. For a simple on/off type controller mechanism 52, the controller mechanism is simply turned from an off to an on position. For a multi-position controller mechanism 52, the mechanism is adjusted by the operator to produce a desired frequency and amplitude of vibration within one or more of the respective vibration devices 50.

At this point, a doctor accesses the dilated veins for the purpose of introducing an IV or for performing a blood draw. The controller mechanism 52 is turned to an off position such that the vibration devices are not vibration. The tourniquet is then removed either by uncoupling the hook portion 32 from the loop portion, as shown in FIGS. 1 and 2, or by uncoupling each of the pegs 60 from the respective holes 60, as shown in FIGS. 3 and 4, and then uncoupling the free end 44 from within the fastening loop 24.

The vibrating tourniquet 20 therefore provides a doctor with the means for accessing dilated veins for the purpose of introducing IV or performing blood draws, and further provides the patient with an additional means for decreasing the sensation of pain when needles or angiocatheters are being introduced by interrupting nerve transmissions and supply external stimulus. The vibrating tourniquet 20 therefore decreases the pain and anxiety associated with blood draws and IV placements.

The vibrating tourniquet 20 therefore provides an improved standard of patient care that is beneficial for hospitals, surgical centers, and doctors'offices. The vibrating tourniquet 20 is easy to produce, and a minimum of additional cost, as compared with traditional tourniquets. Further, the vibrating tourniquet 20 is reusable.

While particular embodiments of the invention have been shown, it will be understood, of course, that the invention is not limited thereto since modifications may be made by those skilled in the art, particularly in light of the foregoing teachings. It is, therefore, contemplated by the appended claims to cover any such modifications as incorporate those features that constitute the essential features of these improvements within the true spirit and the scope of the invention.

What is claimed is:

1. A tourniquet comprising:
a strap portion having a first end and a free end;
a fastening loop coupled to said first end;
a fastening mechanism coupled to said strap portion near said free end;
at least one vibration device coupled to said strap portion between said fastening mechanism and said fastening loop;
a controller mechanism coupled to said strap portion and electrically coupled to said at least one vibration device; and
a power source electrically coupled to said controller mechanism, wherein said controller mechanism can be actuated to send an electrical signal to at least one of said at least one vibration device, said at least one of said at least one vibration device vibrating at a predetermined frequency and a predetermined amplitude as a function of said electrical signal.

2. The tourniquet of claim 1, wherein said fastening mechanism comprises:
a hook portion coupled to said strap portion; and
a loop portion coupled to said strap portion, said hook portion and said loop portion separated by a middle portion of said strap portion.

3. The tourniquet of claim 1, wherein said fastening mechanism comprises:
at least one peg coupled to said strap portion; and
at least one hole coupled to said strap portion between said at least one peg and said free end.

4. The tourniquet of claim 1, wherein said controller mechanism comprises an on/off controller.

5. The tourniquet of claim 1, wherein said frequency and said amplitude are separately controlled by said controller mechanism.

6. The tourniquet of claim 1, wherein said controller mechanism comprises a multi-position controller mechanism, each of said positions of said multi-position controller mechanism sending a unique electrical signal to at least one of said at least one vibration devices, each of said at least one vibration devices vibrating at a preset vibrational frequency and a preset vibrational amplitude in response to said unique electrical signal.

7. The tourniquet of claim 1, wherein said hook portion includes a backing material that is sewn to said strap portion and wherein said look portion includes a backing material that is sewn to said strap portion.

8. The tourniquet of claim 1, wherein said hook portion includes a backing material that is adhesively coupled to said strap portion and wherein said look portion includes a backing material that is adhesively coupled to said strap portion.

9. The tourniquet of claim 2, wherein said hook portion is coupled to said loop portion such that said middle portion of said strap portion is coupled within said fastening loop.

10. The tourniquet of claim 3, wherein one of said at least one pegs is coupled within one of said at least one holes such that said middle portion of said strap portion is coupled within said fastening loop.

11. A method for decreasing pain and anxiety associated with blood draws or IV placements, the method comprising:
(a) forming a vibrating tourniquet by:
providing a strap portion having a first end and a free end;
coupling a fastening loop to said first end;
coupling a fastening mechanism to said strap portion near said free end;
coupling at least one vibration device to said strap portion between said fastening loop and said fastening mechanism;
coupling a controller mechanism to said strap portion between said fastening loop and said fastening mechanism;
electrically coupling said controller mechanism to each respective one of said at least one vibration device; and
coupling an electrical power source to said controller mechanism;
(b) coupling said vibrating tourniquet to an appendage of a patient;
(c) actuating said controller mechanism to send an electrical signal to at least one of said at least one vibration devices, said at least one vibration device vibrating at a preset frequency and a preset amplitude in response to said electrical signal;
(d) performing the blood draw or the IV placement on said patient;
(e) actuating said controller mechanism to cease sending an electrical signal to said at least one of said at least one vibration devices; and
(f) uncoupling said vibrating tourniquet from said appendage.

12. The method of claim 11, wherein coupling a fastening mechanism near said free end comprises:
coupling a hook portion of a hook and loop fastening system to said strap portion near said free end; and
coupling a loop portion of a hook and loop fastening system to said strap portion near said free end, said hook portion separated from said loop portion and defining a middle portion in said strap portion.

13. The method of claim 12, wherein (b) coupling said vibrating tourniquet to an appendage of a patient comprises: (b) coupling said vibrating tourniquet to an appendage of a patient by:
wrapping said vibrating tourniquet around an appendage of a patient;
inserting said free end of said strap through said fastening loop such that said vibrating tourniquet is tightly secured to said appendage to dilate a vein within said appendage and such that said at lease one vibrating device lies adjacent to said appendage; and
fastening said hook portion to said loop portion such that said middle portion is in close contact with and wrapped around said fastening loop.

14. The method of claim 11, wherein coupling a fastening mechanism near said free end comprises:
coupling at least one peg to said strap portion near said free end; and
introducing a hole to said strap portion corresponding in size and shape to one of said at least one pegs at a position between said at least one peg and said free end.

15. The method of claim 14, wherein (b) coupling said vibrating tourniquet to an appendage of a patient comprises: (b) coupling said vibrating tourniquet to an appendage of a patient by:
wrapping said vibrating tourniquet around an appendage of a patient;
inserting said free end of said strap through said fastening loop such that said vibrating tourniquet is tightly secured to said appendage to dilate a vein within said appendage and such that said at lease one vibrating device lies adjacent to said appendage; and
inserting one of said at least one pegs within one of said at least one holes such that said middle portion is in close contact with and wrapped around said fastening loop.

16. The method of claim 11, wherein coupling a controller mechanism to said strap portion comprises coupling an on/off controller mechanism to said strap portion between said fastening loop and said fastening mechanism.

17. The method of claim 11, wherein coupling a controller mechanism to said strap portion comprises coupling a multi-position controller mechanism to said strap portion between said fastening loop and said fastening mechanism.

18. The method of claim 17 further comprising:
(g) adjusting said actuation of said multi-position controller to send a second electrical signal to at least one of said at least one vibration devices, said at least one vibration device vibrating at a second preset frequency and amplitude in response to said second electrical signal, said second preset frequency and said second preset amplitude being different than said preset frequency and preset amplitude.

19. The method of claim 11, wherein coupling a controller mechanism to said strap portion comprises coupling a controller mechanism to said strap portion between said fastening loop and said fastening mechanism, said controller mechanism capable of sending an electrical signal to each of said at least one vibration-devices to separately control an amplitude and a frequency of a vibration generated by each of said at least one vibration devices in response to said electrical signal.

20. The method of claim 17 further comprising:
(g) adjusting said actuation of said multi-position controller to send a second electrical signal to at least one of said at least one vibration devices, said at least one vibration device vibrating at a second preset frequency and a second amplitude in response to said second electrical signal, said second preset frequency being different than said preset frequency.

21. The method of claim 17 further comprising:
(g) adjusting said actuation of said multi-position controller to send a second electrical signal to at least one of said at least one vibration devices, said at least one vibration device vibrating at a second preset frequency and a second preset amplitude in response to said second electrical signal, said second preset amplitude being different than said preset amplitude.
